(19) United States
(12) Patent Application Publication
(10) Pub. No.: US 2025/0194972 A1
(43) Pub. Date: Jun. 19, 2025

(54) POSTPARTUM HEMORRHAGE COLLECTION DEVICE

(71) Applicant: CooperSurgical, Inc., Trumbull, CT (US)

(72) Inventors: Kevin L. Delaney, Bloomington, IN (US); Andrew P. Isch, Lafayette, IN (US); Willie C. McRoy, JR., West Lafayette, IN (US); Ashley Molnar, Coal City, IL (US); Anna Pyo, Indianapolis, IN (US); Donald R. Sandmore, Elletsville, IN (US)

(21) Appl. No.: 18/539,672

(22) Filed: Dec. 14, 2023

(51) Int. Cl. A61B 5/15

(52) U.S. Cl. CPC A61B 5/150045; A61B 5/150366

(57) ABSTRACT

A fluid collection device for a postpartum hemorrhage monitoring system includes a cup comprising an open proximal end, an open distal end, and an interior extending between the open proximal and distal ends. The interior is configured to receive a fluid. The cup comprises an anterior surface configured for placement adjacent an anterior vaginal wall of a patient and a posterior surface configured for placement adjacent a posterior vaginal wall of a patient. A ring extends radially exteriorly from the open proximal end of the cup and is incrementally expandable and contractible. A stem having an open proximal end, an open distal end, and a lumen extending therebetween, extends between the open distal end of the cup and is configured to receive the bodily fluid from the interior of the cup. The ring is movable between a collapsed configuration to an expanded configuration upon receiving a fluid within the ring.

### TECHNICAL FIELD

The present disclosure relates to a collection device for collecting postpartum hemorrhaging. Specifically, the present disclosure relates to a collection device for collection and measurement of postpartum hemorrhaging.

### BACKGROUND

The American College of Obstetricians and Gynecologists (ACOG) defines postpartum hemorrhaging (PPH) as cumulative blood loss greater than or equal to 1,000 ml, or blood loss accompanied by signs or symptoms of hypovolemia within 24 hours after the birth process. PPH is the leading cause of maternal mortality, affecting 5% of women giving birth worldwide. Late recognition and delayed management of PPH have been identified as the main contributing factors to maternal death. Current methods for PPH detection include visual estimation of the blood-soaked mat, gauze, towels, and pads; referencing a chart with images and estimated volumes; weighing pads before and after use; and under buttocks drapes that collect fluid.

Visual estimation is inaccurate and unreliable as it does not improve with specialty, age, or clinical experience. Weighing pads before and after use requires waiting until the pads are blood-soaked, leading to delays in blood loss monitoring. Under buttocks drapes are prone to accidents and low in both quality and accuracy. These methods lead to inaccuracies and late identification of PPH, hindering timely management of PPH. This presents a need for a quantitative, cumulative blood loss monitoring solution that is quick to implement.

### SUMMARY

Offering a single (one size can fit all) device simplifies the issue of consolidated raw material inventory, provides streamlined manufacturing processes, minimizes end user storage space for multiple sizes, and avoids incorrect patient fit and comfort. A single product that can accommodate most, if not all anatomies also would help to avoid time delays where an initial size does provide a leak free fit. Further, having a single, more flexible product to accommodate a larger range of anatomical needs would help to avoid time delays in scenarios where the initial size did not provide a leak free fit resulting incorrect blood loss measurement.

The apparatuses and methods of the present disclosure provide a blood collection device for placement in the female anatomy after giving birth to passively drain hemorrhaging blood into a collection and measurement vessel. The insertable portion of the blood collection system is connected to a drainage tube which in turn is connected to a collection vessel/receptacle. The system provides a single size fluid receiving cup that accommodates variations in anatomy for the rapid identification and accurate measurement of postpartum blood loss after delivery.

The postpartum hemorrhage collection and measurement includes a fluid receiving cup having a substantially a circular open proximal end, an interior configured to receive postpartum hemorrhage, an incrementally expandable and contractable ring extending radially exteriorly from the open proximal end, an anterior surface configured to be placed adjacent an anterior vaginal wall of a patient, a posterior surface configured to be placed adjacent a posterior wall of a patient, an open distal end, a stem having an open proximal end, an open distal end, and lumen therebetween. The expandable ring is incrementally expandable from a collapsed configuration to an expanded configuration upon receiving fluid within the ring and is collapsible upon removal of fluid from the ring. For example, the vaginal canal recovers from delivery, the ring may be inflated or deflated in increments to accommodate changes in the anatomy in a patient.

The fluid receiving cup may also include a pressure point or region adjacent the lip that is designed and configured to facilitate folding of the fluid receiving cup, which may be of a different durometer, thickness, and or material than the rest of the cup. The system may further include drainage tubing extending from the open end of the stent to a free-standing, self-supporting, expandable, fluid collection receptacle. The collection receptacle may be configured to be foldable and/or compressible for packing within packaging, and self-expanding upon release from the package.

One general aspect may include a fluid collection device for a postpartum hemorrhage monitoring system. The fluid collection device may include a cup having an open proximal end, an open distal end, and an interior extending between the open proximal end and the open distal end. The interior may be configured to receive a bodily fluid. The cup may include an anterior surface configured for placement adjacent an anterior vaginal wall of a patient and a posterior surface configured for placement adjacent a posterior vaginal wall of a patient. The device may include a ring extending radially exteriorly from the open proximal end of the cup, where the ring is incrementally expandable and contractible. The device may include a stem having an open proximal end, an open distal end, and a lumen extending therebetween. The stem may extend between the open distal end of the cup and configured to receive the bodily fluid from the interior of the cup. The ring may be movable between a collapsed configuration to an expanded configuration upon receiving a fluid within the ring.

Another general aspect may include a postpartum hemorrhage monitoring device.

The device may include a cup having an open proximal end, an open distal end, and an interior disposed between the open proximal and distal ends. The interior may be configured for receiving a bodily fluid, and the cup may include an anterior surface configured for placement adjacent an anterior vaginal wall of a patient and a posterior surface configured for placement adjacent a posterior vaginal wall of a patient. The device may include a ring extending exteriorly from the open proximal end of the cup. The device may also include a stem having an open proximal end, an open distal end, and a lumen extending therebetween. The stem may extend from the open distal end of the cup and may be configured to receive the bodily fluid from the interior of the cup. The ring may be incrementally inflatable from a collapsed configuration to a first expanded configuration in response to receiving a fluid within the ring, and may be incrementally deflatable from the first expanded configuration to a second configuration upon removal of the fluid from the ring.

Implementations may include one or more of the following aspects.

In accordance with certain aspects of the present disclosure, the anterior surface may taper to the proximal end of the stem.

In another aspect, the posterior surface may be flush with the stem such that the stem is disposed rearward a geometric center of the cup.

In accordance with certain aspects of the present disclosure, the cup may include a pressure point disposed adjacent the ring to facilitate folding of the ring and the cup for placement within a patient.

In some aspects, the pressure point may be in an area of the cup that is thinner than a remainder of the cup.

In some aspects, a pressure point may be disposed in an area of the cup having a durometer less than a durometer of the remainder of the cup.

In some aspects, the pressure point may be disposed adjacent the ring to facilitate folding of the ring and the cup for placement within a patient.

In some aspects, an inflation lumen may be configured to provide inflation fluid to the ring.

In some aspects, the inflation lumen may be integrally formed with the cup.

In some aspects, the inflation lumen may be disposed within a sidewall of the cup.

In some aspects, the cup may have a smooth outer surface adjacent the inflation lumen.

In some aspects, the inflation lumen may extend from the ring to one of the open proximal end and the open distal end of the stem.

In some aspects, the ring may include a superabsorbent material.

In some aspects, the ring may be configured to expand upon contact with the bodily fluid.

In some aspects, the ring may be an expandable balloon.

In some aspects, the ring may be asymmetrical.

In some aspects, the cup may include a pressure point that is disposed adjacent to the ring to facilitate folding of the ring and the cup for placement within a patient.

In some aspects, the pressure point may include an indentation.

### BRIEF DESCRIPTION OF THE DRAWINGS

FIG. 1 is an example of a blood loss monitoring system/kit assembled in accordance with the teachings of the present disclosure;

FIG. 2 shows an example blood loss monitoring system/kit system with a fluid receiving cup having an expandable rim assembled in accordance with the teachings of the present disclosure;

FIG. 3 shows an end view of the fluid receiving cup shown in FIG. 2;

FIG. 4 shows a partial end view of a different example fluid receiving cup that can be used with the system of FIGS. 1 and 2;

FIG. 5 shows a side view of the fluid receiving cup of FIGS. 2 and 3;

FIG. 6 shows a side view of a different example fluid receiving cup that can be used with the system of FIGS. 1 and 2;

FIG. 7 shows a cross sectional view of a different example fluid receiving cup that can be used with the system of FIGS. 1 and 2;

FIG. 8 shows a cross section view of a different example fluid receiving cup that can be used with the system of FIGS. 1 and 2;

FIG. 9 shows a front view of the fluid receiving cup of FIG. 8;

FIG. 10 shows a partial side view of a different example fluid receiving cup that can be used with the system of FIGS. 1 and 2, and having an expandable rim in an unexpanded state;

FIG. 11 shows a partial side view of the fluid receiving cup of FIG. 10 with the rim in an expanded state;

FIG. 12 shows a partial cross-sectional view of the fluid receiving cup of FIG. 10;

FIG. 13 shows a magnified cross-sectional view of the rim of the fluid receiving cup of FIG. 12;

FIG. 14 shows a partial cross-sectional view of the fluid receiving cup of FIG. 11 with the rim in the expanded state;

FIG. 15 is perspective view of a different example fluid receiving cup that can be used with the system of FIGS. 1 and 2;

FIG. 16 is a perspective view of a different example fluid receiving cup that can be used with the system of FIGS. 1 and 2;

FIG. 17 is a front view of a different example fluid receiving cup that can be used with the system of FIGS. 1 and 2;

FIG. 18 is a perspective view of the cup of FIG. 17.

FIG. 19 is a front view of a different example fluid receiving cup that can be used with the system of FIGS. 1 and 2;

FIG. 20 is a perspective view of the fluid receiving cup of FIG. 19;

FIGS. 21-23 are views of the cups of FIGS. 15, 17, and 19 having shorter stems;

FIGS. 24A-C are views of a different example fluid receiving cup that can be used with the system of FIGS. 1 and 2;

FIGS. 25A-B are views of a different example fluid receiving cup that can be used with the system of FIGS. 1 and 2;

FIGS. 26A-B are views of a different example fluid receiving cup that can be used with the system of FIGS. 1 and 2, the cup having a curved stem;

FIGS. 27 and 28 show a technician folding of any of the cups of the present disclosure for insertion;

FIG. 29 shows a different fluid collection system with the fluid collection cup of FIGS. 26A-B, showing the folded cup outside of a patient; and

FIG. 30 shows the system of FIG. 29 with the cup placed in the patient's anatomy.

### DETAILED DESCRIPTION

The present disclosure provides a blood loss fluid receiving cup that is part of a blood loss collection system for the measurement and timely identification PPH with a single fluid receiving cup configured to accommodate most if not all anatomical variations between patients and within a single patient. Specifically, the present disclosure provides a single product which addresses the need for multiple sizes of fluid receiving cup diameters to cover a broad range of vaginal canal diameters and vaginal tissue compliance/elasticity.

After a vaginal delivery the vaginal canal anatomy will rebound over time and during the blood loss monitoring period. An expandable/inflatable retention ring allows for real time adjustment and customized fit of a fluid receiving cup that can accommodate changes in the vaginal anatomy during the measurement period as well as the variable anatomies of different patients. Hence, additional sizes are not necessary and the process is streamlined. The fluid receiving cup is a funnel shaped device that is configured to be placed in the vaginal canal soon after delivery of a baby and placenta. It is intended and configured to seal against the wall of the vaginal canal and provide a pathway for blood to flow out of the patient, through a center lumen and drainage tubing, and to a fluid collection receptacle. This allows for accurate quantification of the blood lost from the patient during a postpartum bleeding event. Specifically, the fluid receiving cup is placed intra-vaginally approximately halfway between the introitus and the cervix following the delivery of the placenta after birth takes place.

As used herein, the terms “cup,” “receptacle,” and “funnel” may be used interchangeably and mean the same feature.

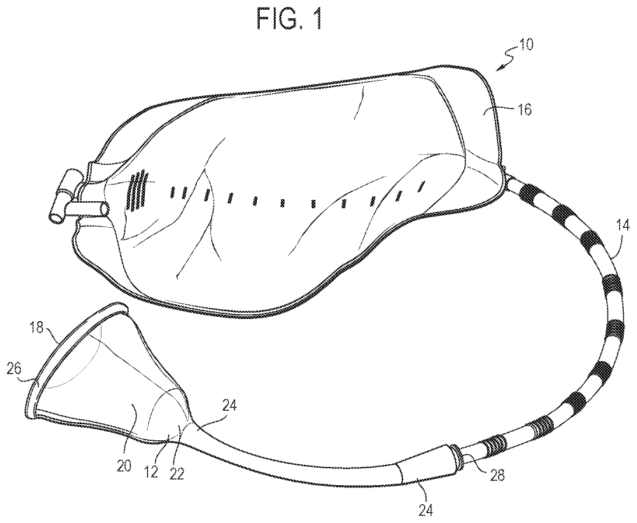

FIG. 1 shows a blood loss collection and measurement system 10. The system 10 includes a fluid receiving cup/funnel 12, drainage tubing 14, and a drainage receptacle 16 connected to the cup 12 via the tubing 14. In another example, the drainage receptacle 16 is free-standing, self-supporting, and expandable. The fluid receiving cup/funnel 12 has a first open end 18, a funnel body 20, a second open end 22 leading to a stem 24 extending from the second open end 22, and a connector 28 for connection the stem 24 to the tubing 14. The cup 12 is configured to receive fluid from a patient experiencing postpartum hemorrhaging, which flows through the cup 12, the lumen of the connector, through the tubing 14, and into the drainage receptacle 16 to be measured.

The fluid receiving cup of examples described herein funnels blood through the drainage tubing into a fluid collection receptacle secured at the side of the patient's bed. This solution provides both quantitative and cumulative blood loss measurement and PPH can be identified more accurately and rapidly than current solutions. The fluid collection receptacle will be labeled at increments of 50 ml or 100 ml to allow physicians to actively monitor blood loss and respond when the PPH threshold has been crossed. Additionally, the fluid collection receptacle will be sealed and secured at the bedside making this solution less prone to accidents or spilling when compared to current monitoring methods. In other embodiments, the receptacle is free standing and may be placed on the floor near the bed side or on another surface.

At the first open end 18, a retention ring/rim 26 surrounds the opening 18 that is configured to be sealingly engaged within the anatomy of the patient. The stem 24 includes a connector 28 that is configured to be attached to the tubing 14. The connection may be with a friction fit conical connector. As used herein the terms “retention ring,” “ring,” and “rim” are used interchangeably and mean the same thing. In some examples, the ring may also serve as a “lip,” as described herein. The fluid collection receptacle 12 is designed and configured to be flexible, free-standing, expandable, and/or compressible for packaging.

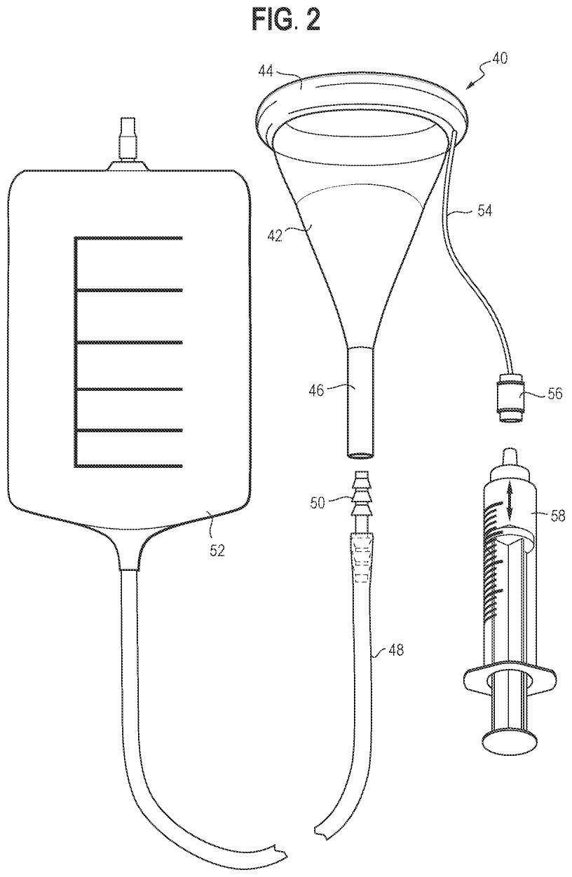

FIG. 2 shows an exemplary system 40 having a fluid collection cup 42 with a variable size retention ring 44. In this embodiment the retention ring 44 is inflatable. The retention ring 44 provides an adjustable and customizable fit within a vaginal canal of a patient. The cup 42 will have a partially to fully circumferential ring 44 at the opening of the cup 42. As shown in FIG. 2, the system 40 includes a generally funnel-shaped cup 42 having a circumferential retention ring 44 and an integral drain tube/stem 46.

The fluid receiving cup 42 may be constructed from soft, medical-grade silicone and is free from phthalate, latex, dioxin, & BPA. The durometer may range from 40 A to 70 A and, in some examples, is from about 50 A to 60 A.

The stem/integral drainage tube 46 is attached to drainage tubing 48 via connector 50. As shown in FIG. 2, the connector 50 is a barbed attachment. Further attachment systems may include a friction fit, snap fit, threaded fit, luer-lock fit, or other suitable attachment systems. The tubing 48 is connected to a collection receptacle 52 for receipt and measurement of blood loss. The retention rim or ring 44 may be a balloon or a balloon like structure. The ring/balloon 44 will have an inflation line 54, which allows for fluid to be injected to pressurize and expand the ring 44. The inflation line 54 may have an inflation check valve 56 for inflation and deflation. Fluid is introduced into the line 54 through the valve 56 via an inflation syringe 58, for example. The ring/balloon may be integral with the funnel portion of the cup 42 or may be bonded to the cup 42 at the opening. The inflation line 54 may be attached to the integral drainage tube 48. Inflation of the ring/balloon 44 may expand radially in a range of approximately 0.25 cm to approximately 4.0 cm (e.g., in a range of approximately 0.75 cm to approximately 2.5 cm) to increase the overall diameter of the ring 44. While the ring 44 itself is inflatable, the remainder of the cup is not.

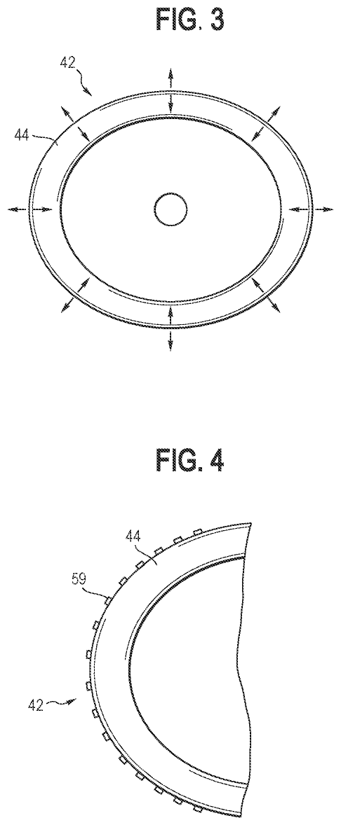

The fluid receiving cup 42 is configured to be placed in the anatomy. The clinician inflates the ring 44, if necessary, to the appropriate size the patient to provide a leak proof seal around the edge of the cup 42. FIG. 3 shows a front view of the cup 42. The arrows indicate inflation/deflation of the ring 44 wherein such inflation/deflation causes the ring 44 to conform to the patient's anatomy, thereby improving patient comfort, sealing, and adjustment to the surrounding tissue. FIG. 4 is a partial view of the ring/balloon 44 of FIG. 3. The outer surface of the ring/balloon 44 may be smooth. Alternatively, as shown in FIG. 4, the ring/balloon 44 may include retention ribs 59 on the ring 44 partially or fully about the outer perimeter. Other examples may include texturing on the surface of the ring 44.

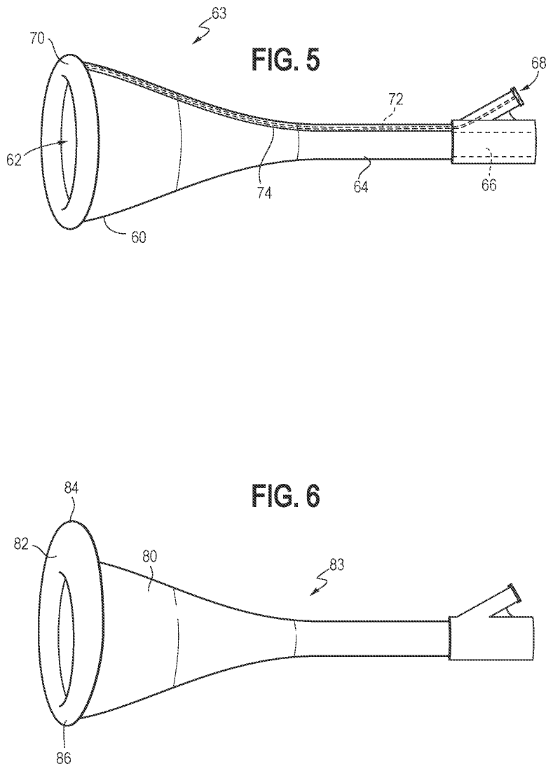

FIGS. 5 and 6 show side views of example fluid collection devices 63, 83 assembled in accordance with the teachings of the present disclosure. As shown in FIG. 5, the collection device 63 includes a cup 60 having a funnel body, an open proximal/first end 62, a stem 64 extending from the body to provide a fluid passage way from the open proximal end 62, through the stem 64, and through the drainage tubing 66 attached to the distal end of the stem 64. As shown in FIG. 5, the drainage tubing 66 is integrally formed with an inflation port 68. The cup device 63 includes an inflatable retention ring 70. An inflation line 72 extends from the inflation port 68 to provide inflation fluid to the retention ring 70. As shown, the inflation line 72 is disposed within a sidewall 74 of the fluid receiving cup 60 and sidewall of the stem 64. In one example, the cup 60 and inflation line 72 may be manufactured as a two-lumen tubing with the fluid receiving cup 60 formed during a secondary flaring process. Forming the inflation line 72 within the wall 74 of the cup 60 and stem 64 may provide a smooth internal and external profile to improve fluid flow, sealing within a patient, and patient comfort. The inflation line 72 may also provide a reference to the clinician for the fold location when deploying or removing the cup device 63 from the patient. The inflation line 72 may also provide a rotational reference as to which side of the cup 60 is intended to be placed anteriorly and which side is to be placed posteriorly.

The expandable ring 70 of the device 63 is symmetrical, however, in other examples, such as the example of FIG. 6, a ring 82 of a different fluid collection device 83 is asymmetrical. The ring 82 is an inflatable balloon, and is not uniformly inflated about the circumference of the proximal opening of the cup 80. Rather, only a portion on the anterior side 84 is inflated, and the posterior side 86 is not inflated or inflated to the same extent as the anterior side 84. For example, only half or two thirds of the inflatable ring 82 is inflated. The inflated portion of the ring 82 is configured to be placed on the anterior side of the vaginal canal. When inflated, the ring 82 pushes the cup 80 toward the posterior side of the vaginal canal to improve drainage and reduce blood pooling and/or clot formation. Alternatively, the posterior side 86 may be inflated to a greater degree than the anterior side 84.

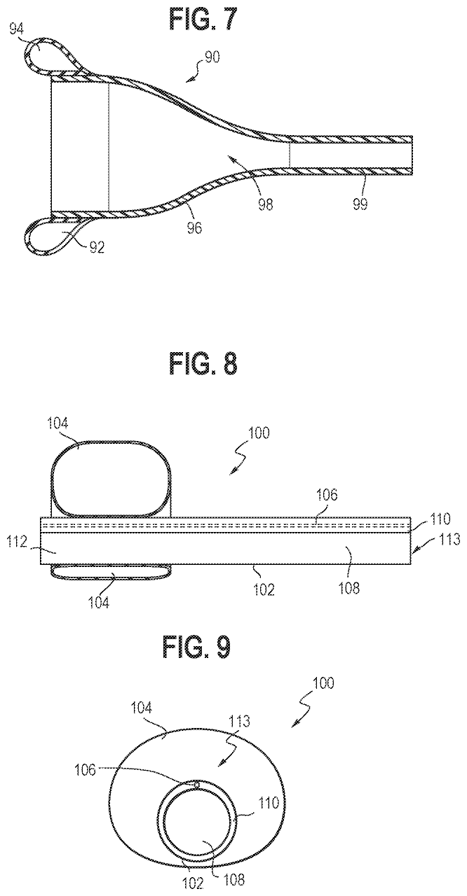

FIG. 7 shows a side cross sectional view of another example cup device 90 for receiving postpartum hemorrhaging. Here, the balloon/ring 92 is a fully circumferential ring which has a tear-drop shaped cross-section. The balloon/ring 92 has a lobe 94 at a distal edge and then tapers to the funnel towards the proximal side of the balloon. The lobe geometry may provide a smooth transition from the vaginal wall to the funnel portion 96 of the cup device 90 and may direct blood flow into an interior cavity 98 of the cup device 90, leading to the stem 99. This example may help reduce blood pooling and clot formation at the tissue to balloon interface.

With all of the examples having an inflatable rim/ring, the physician/clinician can customize the size of the ring/rim and can incrementally inflate and deflate the ring to accommodate a patient's anatomy as the anatomy changes over time postpartum and differs by patient. Further, deflation of the ring/rim may make removal of the cup from the patient easier and more comfortable.

FIG. 8 shows another example of a fluid receiving device 100. A tubing 102, without a flared funnel-shaped body as the previous examples, is provided and can be fitted with a larger inflatable ring 104. Inflation lumen 106 runs side-by-side with the drainage lumen 108. As shown, the inflation lumen 106 is disposed in a sidewall 110 of the drainage tubing 102 to provide a dual-lumen device 100. This design may simplify the manufacturing process and a non-uniform balloon may be incorporated to help place a distal end 112 of the drainage tube at a target location within the patient. FIG. 9 shows an end view of the example of FIG. 8 where the drainage tube lumen 108 is offset from the center of the inflatable ring 104 and the inflation lumen 106 is within the sidewall 110 of the drainage tube 1402. With each of the inflatable examples, the ring is controllably expanded by the physician/clinician.

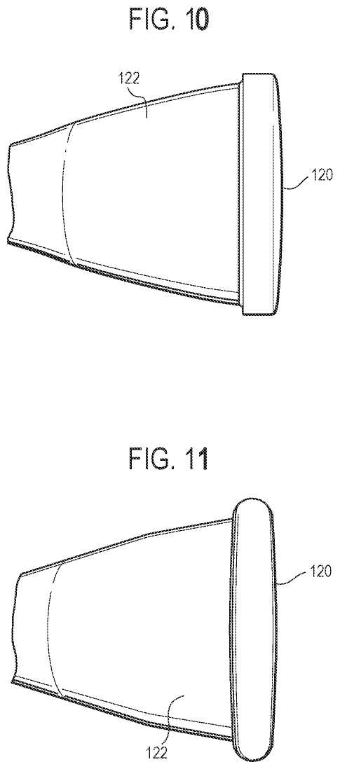

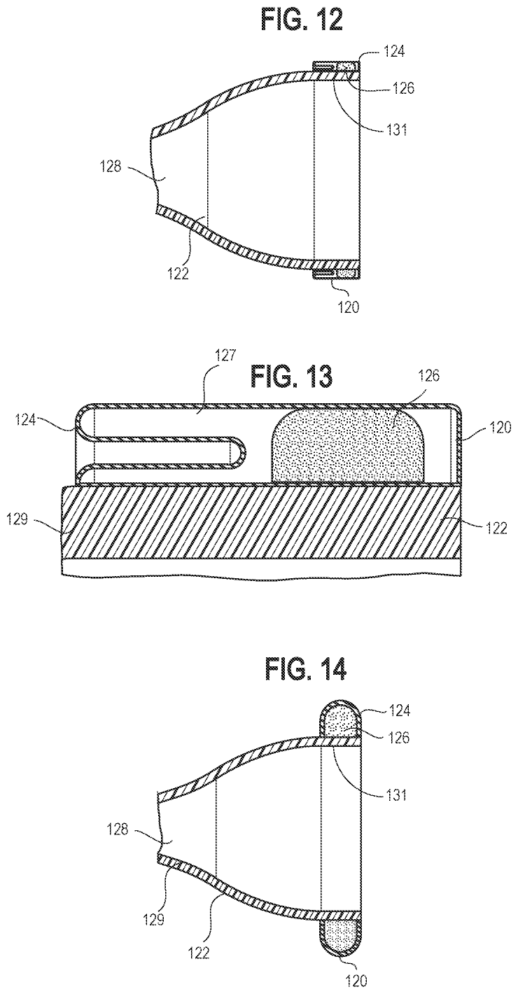

FIGS. 10-14 show an alternative example of a fluid receiving cup 122 having an expandable ring 120. In this example, a ring 120 of the fluid receiving cup 122 expands radially when a filling material 126 absorbs fluid. The ring 120 includes a material that absorbs blood and/or other fluid from the vaginal canal and expands radially outwardly. FIGS. 10, 12, and 13 show the cup 122 with the ring 120 in an initial dry configuration, prior to insertion and absorption of fluid. FIGS. 11 and 14 show the cup 122 with the ring 120 in an expanded configuration after fluid absorption. The expansion provides a custom fit within the patient and a seal between the cup 122 and the patient to prevent blood from leaking past an outer, circumferential edge of the cup 122. The absorbent material 126 may be a sodium polyacrylate (also known as a superabsorbent material) with high absorbent properties. The material 126 may also be a medical grade open cell silicone foam, or other similar material with high expansion rates. The absorbent material 126 may also include one or more of cotton, viscose rayon, or a blend of the same or similar materials that are absorbent and biocompatible. As shown in FIGS. 12-14, the absorbent material 126 is disposed in a thin, porous film layer 124 constructed of mesh or other fluid absorbing material, such as a nonwoven material in menstrual products and diapers. The film layer 124 is bonded to the fluid receiving cup 122 on an outer surface. FIGS. 12 and 13 illustrates the ring 120 in a collapsed state prior to insertion and absorption of fluid.

FIG. 13 is a magnified, cross-sectional view of the cup 122 of FIG. 12 with the ring 120 in a collapsed state prior to insertion. FIG. 14 is a cross-sectional view showing the ring 120 in an expanded state. The absorbent material 126 is configured to interact with fluid and expand, thereby expanding the profile of the ring 120. The expansion may occur both longitudinally and radially to fill out a cavity 127 between the film layer 124 and a wall 129 of the cup 122, as shown in FIG. 13. The film layer 124 will become taut as maximum expansion is reached, thereby limiting the extent to which the retention ring 120 can expand. The expansion will allow the retention ring 120 to contact the surrounding tissue and apply pressure. The applied pressure will also be limited by the extent the film layer 124 can expand. The surrounding tissue will apply a resisting force in the opposing direction to the expansion. The resisting force may be greater than the force from the retention ring 120 and may also compress an opening 131 of the fluid receiving cup 122 radially inwardly. However, a lumen 128 defined by the cup 122 will not completely close due to the limited maximum expansion. Additionally, a seal will be formed between the retention ring 120 and the surrounding tissue to prevent additional fluid from leaking past the retention ring 120. This will direct the blood flow down the central lumen 128 of the cup 122 to be quantified in the collection receptacle. This example provides a single product that may accommodate a large range of patient anatomies, and does not require multiple parts to be manufactured and stocked. This example also provides a custom fit for the patient, as it will expand to conform to the vaginal walls of the patient.

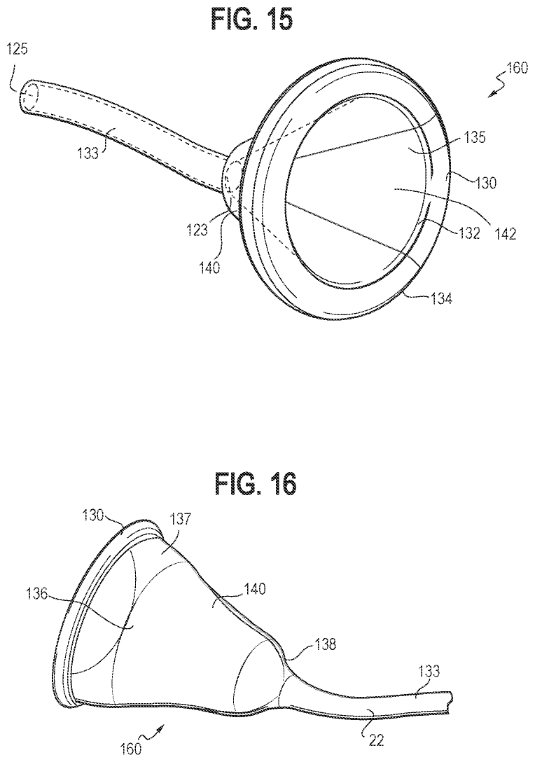

FIGS. 15 and 16 show an example fluid receiving cup device 160 having a retention ring 130, which may have the expandable features in accordance with the teachings of the present disclosure. In one example, the ring 130 may slope inwardly from an outer-most edge 134 to an inner-most edge 132. In another example, the ring 130 may slope inwardly from the inner-most edge 132 to the outer-most edge 134. The ring 130 extends outwardly and uniformly from an exterior sidewall 137 of a funnel 140 of the fluid receiving cup device 160 at an opening 142, but is flush with an interior sidewall 135 of the fluid receiving cup so as not to extend into the interior of the cup device 160. This keeps the interior surface of the fluid receiving cup device 160 unobstructed and unimpeded from any structure that would interfere with the laminar flow of fluid through and out of the cup device 160. A smooth interior surface also avoids pooling and clotting of blood, which can lead to inaccurate measurement or obstruction of the fluid flow.

As shown in FIG. 16, an area 136 adjacent to and extending distally from the ring 130 may have a thinner section that allows the funnel body 140 of the cup device 160 to easily fold on itself for insertion into a patient. This thinner section 136 is located in the upper half of the funnel body 140 of the fluid receiving cup 160, and in some examples, in the upper third of the funnel body 140 of the fluid receiving cup 160. The thinner section 136 gradually thickens both circumferentially and axially along the fluid receiving cup 160 toward the stem 133. The fluid receiving cup 160 has its maximum thickness 90° circumferentially from the thinner section 136 and at a base 138 of the funneling portion 140 of the fluid receiving cup 160. The funneling portion 140 of the fluid receiving cup 160 is contoured on one side while narrowing to the base 138 to provide additional anchoring as the vaginal walls collapse.

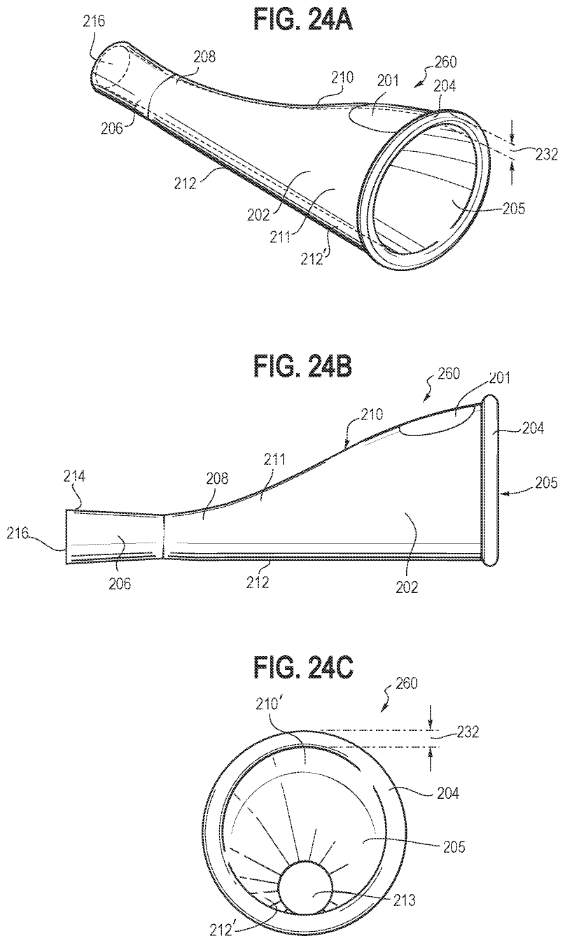

A portion (shown as element 232 in FIGS. 24A and C and 25A and C) of the ring 130 at the top of the ring and adjacent the thinner section 136 also may be thinner than the rest of the ring 130 to further facilitate folding of the fluid receiving cup 160. In place of or in addition to the thinner section, the fluid receiving cup device 160 may also include a pressure point or region (shown as element 201, in FIGS. 24A-B, and 25A-B) on the anterior surface of the fluid receiving cup adjacent the ring 130. The pressure point 201 or region may be a region of the sidewall 135 that is of thinner material, of a different durometer, and/or of a different material. The pressure point or region is located in the upper half, and in some examples an upper third, of the funnel body 140 of the fluid receiving cup device 160.

As shown, a stem 133 extends between an open end 123 of the base 138 and an open end 125 of the stem 133, and is designed and configured to be attached to the drainage tubing 14 for transporting fluid to a fluid collection receptacle 12 for monitoring and measurement. In some examples, the fluid collection funnel device 160 is constructed from soft, medical-grade silicone and is free from phthalate, latex, dioxin, & BPA. The durometer may range from 40 A to 70 A and, in some examples, is from about 50 A to 60 A. The thinner section 136 and/or pressure point 201 or region may have of a lower durometer and in the range of about 20 A to 40 A.

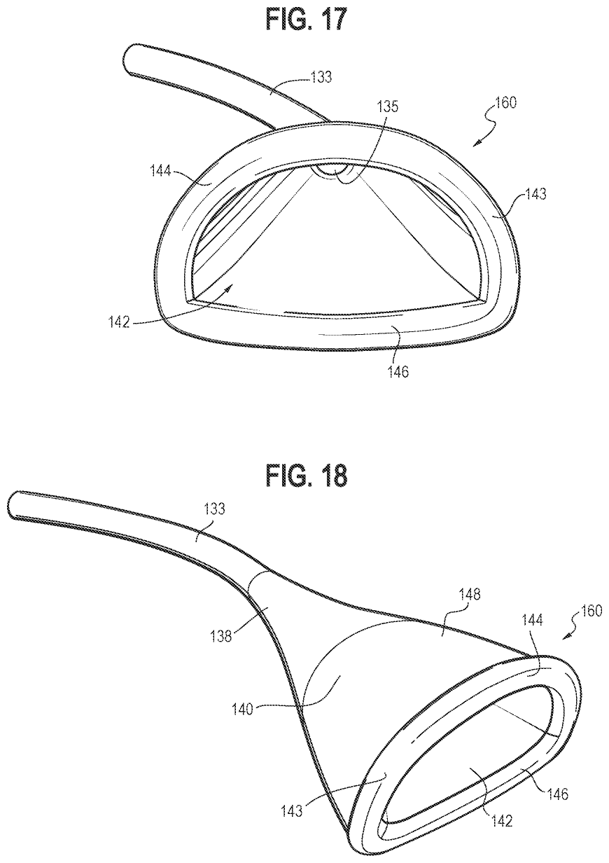

FIGS. 17 and 18 show another example fluid collection funnel device 160 having a noncircular contour. As shown, the device 160 includes a funnel body 140 and a stem 133. A mouth 142 of the fluid collection funnel device 160 has a semicircular contour 144 with a shallow arc contour 146. The different contour shapes 144, 146 may increase the ergonomics and comfort of the device 160. A protruding lip 143 creates a firm anchoring feature with which the fluid collection funnel device 160 can press against the vaginal walls. A wall section 148 of the fluid collection funnel device 160 adjacent and extending from the protruding lip 143 may be thinner that allows the device to easily fold on itself for insertion. The thinner wall section 148 gradually thickens both circumferentially and axially along the semicircular portion 144 of the device 160, thereby achieving a maximum thickness at an intersection of the semicircular contour 144 and shallow arc contour 146 as well as at the base 138 of the funnel body 140. As shown, the funnel body 140 is contoured while narrowing to the base 138 to provide some additional anchoring as the vaginal walls collapse. As described above, the protruding lip 143 adjacent the thinner section 148 also may be thinner than the rest of the protruding lip 143 to further facilitate folding of the fluid collection funnel device 160. In place of or in addition to the thinner section 148, the fluid collection funnel device 160 may also include a pressure point or region (element 201, as shown in FIGS. 24A-B and 25A-B) on the anterior surface of the fluid collection funnel device 160 adjacent the protruding lip 143. Each of the features described with regard to FIGS. 1-14 may also be included or coupled to the device 160 of this example.

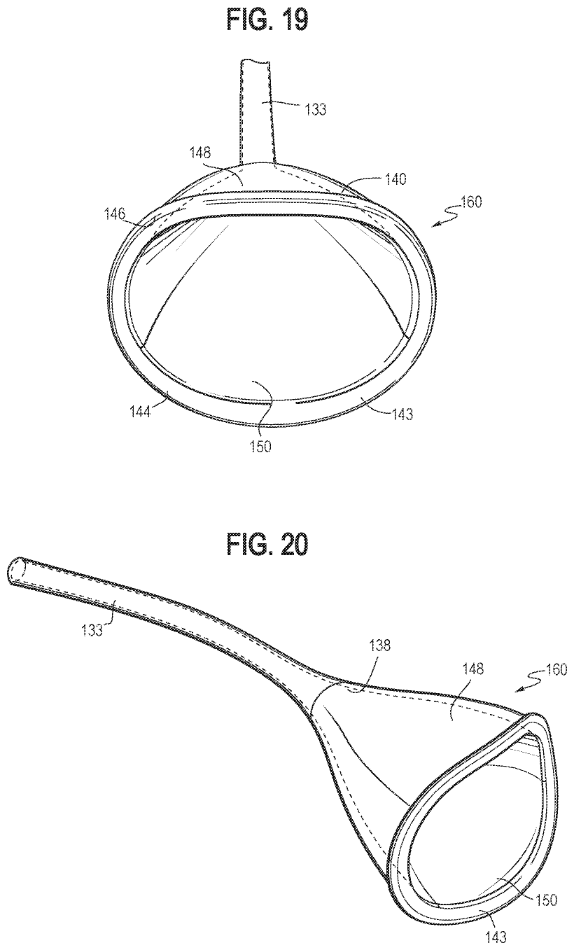

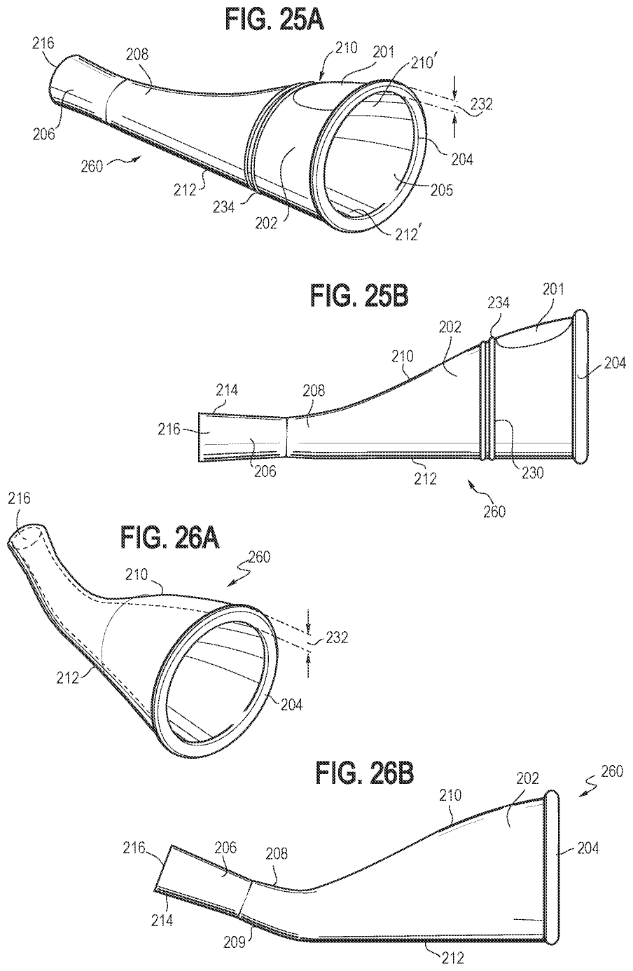

FIGS. 19 and 20 show another fluid collection funnel device 160 having a noncircular contour, but with a distal extension 150. The fluid collection funnel device 160 in this example has a semicircular contour 144 and a shallow arc contour 146. The different contour shapes 144, 146 may increase the ergonomics and comfort of the device 160. A protruding lip 143 creates a firm anchoring feature with which the fluid collection funnel device 160 can press against the vaginal walls. The shallow arc contour 146 also forms a distal extension 150 designed to follow the angle of the vaginal canal relative to the uterus. As described above, the wall portion 148 of the fluid collection funnel device 160 adjacent the protruding lip 143 may be thinner to allow the device 160 to easily fold on itself for insertion. The thinner section gradually thickens both circumferentially and axially along the semicircular portion 144 of the device 160 and the fluid collection funnel device 160 achieves its maximum thickness at the intersection of the semicircular contour 144 and shallow arc contour 146 as well as at the base 138 of the funnel body 140 of the device 160. The funnel body 140 is contoured while narrowing to the base 138 to provide some additional anchoring as the vaginal walls collapse. As described above a portion of the protruding lip 143 adjacent the thinner section also may be thinner than the rest of the protruding lip 143 to further facilitate folding of the fluid collection funnel device 160. In place of or in addition to the thinner section, the fluid collection funnel device 160 may also include a pressure point (as shown in FIGS. 24A and 25A) on the anterior surface of the fluid collection funnel device 160 adjacent the protruding lip 30.

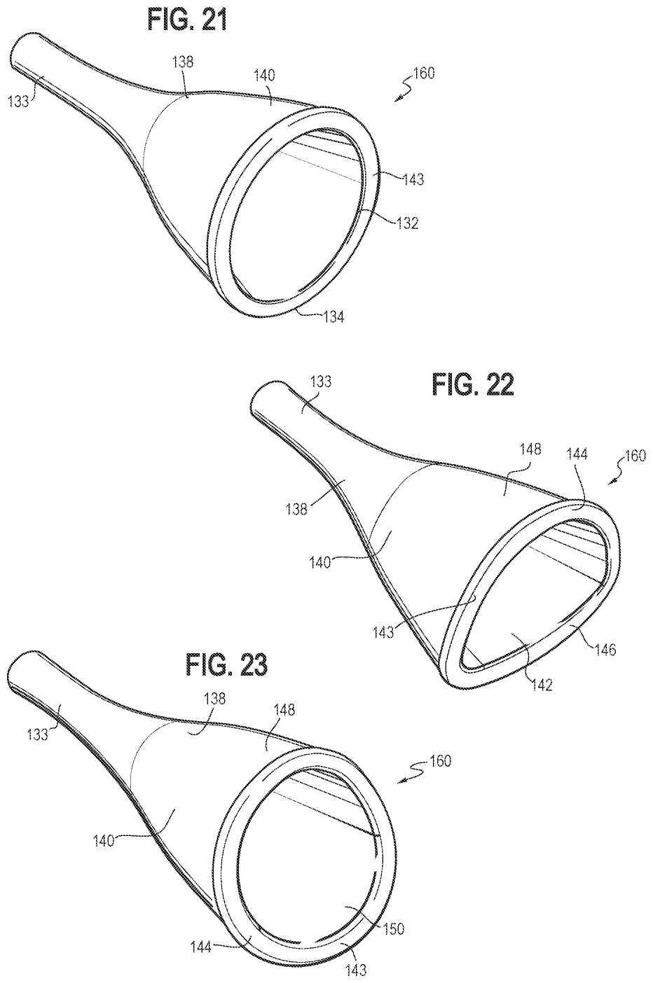

FIGS. 21-23 show fluid collection funnel devices 160 of FIGS. 15-20 with shorter stems 133. As shown and more fully described above with reference to FIGS. 15-20, each fluid collection funnel has a funneling portion 140, a protruding lip 143 and is configured and designed to be connected to drainage tubing which in turn is connected to a fluid collection receptacle.

FIGS. 24A-C show another example of a fluid collection funnel device 260. FIG. 24A shows a fluid collection funnel device 260 having a funnel body 202, a symmetrically circular lip 204, a symmetrically circular proximal opening 205, and a stem 206 extending from a base 208 of the funnel body 202. The funnel body 202 has an anterior surface 210 and a posterior surface 212 opposite the anterior surface 210. As shown in FIG. 24B, the anterior surface 210 tapers from the symmetrically circular lip 204 to the base 208 of the funnel body 202. An opening 213 shown in FIG. 24C is provided in the base 208 of the fluid collection funnel device 260, which leads into stem 206. The distal end of the stem 206 has a distal opening 216. Returning to FIG. 24B, the anterior surface 210 has a continuous and contoured taper extending from the lip 204 to the base 208. The stem 206 extending from the base 208 has a slight flare 214 at its end 216 to receive a connector of the tubing 14.

The posterior surface 212 extending from the lip 204 to the base 208 has little or no taper, thereby providing a flattened or laminar flow path within the interior of the funnel device 260 from the symmetrically circular opening 205 to and through the stem 206 to prevent pooling of blood in the funnel body 202 of the funnel device 260 when the patient is lying supine or semi-supine after giving birth, thereby preventing inaccuracies in hemorrhaging measurement. The lip 204 protrudes uniformly from the exterior wall 211 at the opening 205, but does not extend into the interior of the funnel device 260 thereby avoiding any obstruction or impeding of fluid flow. The interior surface of the funnel presents a smooth and uniform surface free of any protrusions or other obstruction.

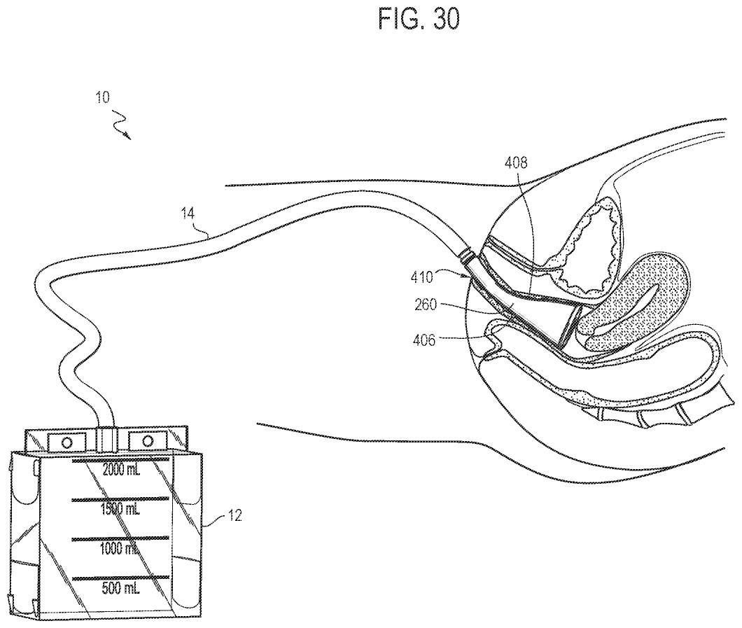

As shown in FIGS. 24A-B and 25A-B, the posterior surface 212 has little or no taper from the lip 204 to the base 208 and therefore no bulge in which blood can pool in the interior. The stem 206 extends from the base 208 and has a slight flare 214 toward its open distal end 216 to facilitate attachment to a connector (not shown), for example a barb connector, of a drainage tube, such as the drainage tube 14 of FIG. 1. As shown in FIGS. 24A-C, the stem 206 of the funnel device 260 is substantially flush with the posterior surface 212 of the funnel body 202 and is offset from the geometric center of the funnel device 260 to provide the flattened flow path in the interior of the funnel device 260. FIG. 24C shows a front view of the funnel device 260, and the interior of the funnel device 260, having an internal anterior surface 210′ and an interior posterior surface 212′. As shown in FIG. 24B, the stem 206, except for the slight taper 214 at the distal end 216, is substantially flush with the posterior surface 212. Hence, the interior posterior surface 212′ of the funnel body 202 is substantially flush with the interior posterior surface 212′ of the stem 206 such that fluid flow from the opening 205 to and through the open distal end 216 is flat and laminar. The interior posterior surface 212′ has little, or in some examples no, taper from the opening 205 to and through the base 208. This shape of the fluid collection funnel device 260 is designed and configured such that when the funnel device 260 is inserted into a patient, as shown in FIG. 30, the anterior surface 210 of the funnel device 260 engages and conforms to the anterior vaginal wall 218 and the posterior surface 212 engages and conforms to the posterior vaginal wall 220 and fluid is prevented from pooling in the funnel device 260.

In the examples of FIGS. 24A-C and 25A-B, the fluid collection funnel 260 has a pressure point 201 or section adjacent the lip 204 of the funnel device 260 and extending from the lip 204. In some examples, the pressure point 201 is directly adjacent the lip 204. The pressure point 201 or section has, as described above, a lesser thickness of material compared to other portions of the funnel device 260. Additionally or optionally, the pressure point 201 has a lower durometer than other portions of the funnel device 260 to facilitate folding, or may be a different, more pliable material to facilitate folding. As shown in FIGS. 24A and 25A, a portion 232 of the lip 204 of the funnel device 260 at the top of the funnel adjacent the pressure or folding section 201 is thinner than other portions of the lip 204, which also facilitates folding. While the lip 204 in this region may be thinner than the rest of the lip 204, the lip 204 and the opening 205 both remain symmetrically circular in these examples. As shown in FIGS. 25A and B, the funnel body 202 may include grips 234, such as, for example, ridges, dimples, grooves, etc., on an outer surface to facilitate removal of the funnel device 260. Ridges or other gripping features may be provided on and along the stem 206, as well, to facilitate removal.

FIGS. 26A and B shown a further example of a fluid collection funnel device 260. In this example, the fluid collection funnel 260 has similar features to the funnel described with regard to FIGS. 24 and 25 above, but includes a pre-curved end 209 at the base 208 to conform to a patient's anatomy. As shown, the posterior surface 212 (as well as the interior posterior surface 212′) has no taper and provides a flattened, unobstructed flow path from the opening 205 to the base 208.

The fluid collection funnel devices 260 of FIGS. 24A-26B may be constructed from soft, medical-grade silicone and is free from phthalate, latex, dioxin, & BPA. The durometer may range from 40 A to 70 A and, in some examples, is from about 50 A to 60 A. The thinner section and/or pressure point or region may be of a lower durometer and in the range of about 20 A to 40 A. Each of the features described with regard to FIGS. 1-14 may also be included in the examples of FIGS. 15-26B.

In FIGS. 15-26B, the funnel devices 160, 260 are shaped and configured to enter the anatomy following birth and anchor within the vagina. The protruding lip 143, 204, 330 of each funnel device 160, 260 creates a firm anchoring feature with which the insertable portion of the device can press against the vaginal walls. The interior-most edge of the lip 30, 204 may not be concentric with the exterior most edge and may slope inwardly or outwardly, as described above. The funnel device 260 of the examples shown in FIGS. 24A-25B may have a thinner section gradually thickening both circumferentially and axially along the insertable portion of the device and achieving maximum thickness 90° circumferentially from the thinner section and at the base of the funnel body of the device. As described above, the funnel body contoured on its anterior surface while narrowing to the base to provide some additional anchoring as the vaginal walls collapse. Each of the features described with regard to FIGS. 1-14 may be included in the any of the examples of FIGS. 24A-26B. For example, any of the protruding lips 143, 204, 330 described herein may be incrementally expandable and collapsible.

The fluid collection cups and devices as described herein may have a dimension in a range of about 3 to about 6 inches in length from the opening to the distal end of the stem, and in some examples, of about 4 to about 5 inches in length from the opening to the distal end of the stem. The inner diameter of fluid collection funnel at the opening has a dimension in a range of about 1.5 to about 2.5 inches, and in some examples, of about 1.25 to about 2.25 inches. The thickness of the lip from top to bottom is in a range of about 0.1 inches to about 0.2 inches, and in some examples, of about 0.13 to about 0.17 inches, and in some examples, about 0.15 inches. The outer diameter of the fluid collection funnel at the opening has a dimension in a range of about 1.5 to about 2.5 inches, and in some examples, of about 1.25 to about 2.25 inches, and in some examples, of about 1.7 to about 1.9 inches. The angle of the taper at the distal opening of the stem has a dimension in a range of about 4.5° to about 6.5°, and in some examples, of about 5° to about 6°. The inner diameter of the base of the fluid collection funnel has a dimension in a range of about 0.2 to about 0.35 inches, and in some examples, of about 0.25 to about 0.3 inches. The radius of the lip has a dimension in a range of about 0.05 inches to about 0.10 inches, and in some examples, of 0.07 to about 0.08 inches, and in some examples, about 0.075 inches. The dimensions are an approximate of the female human anatomy to provide the most effective and comfortable fit for the patient and for accurate fluid loss measurement.

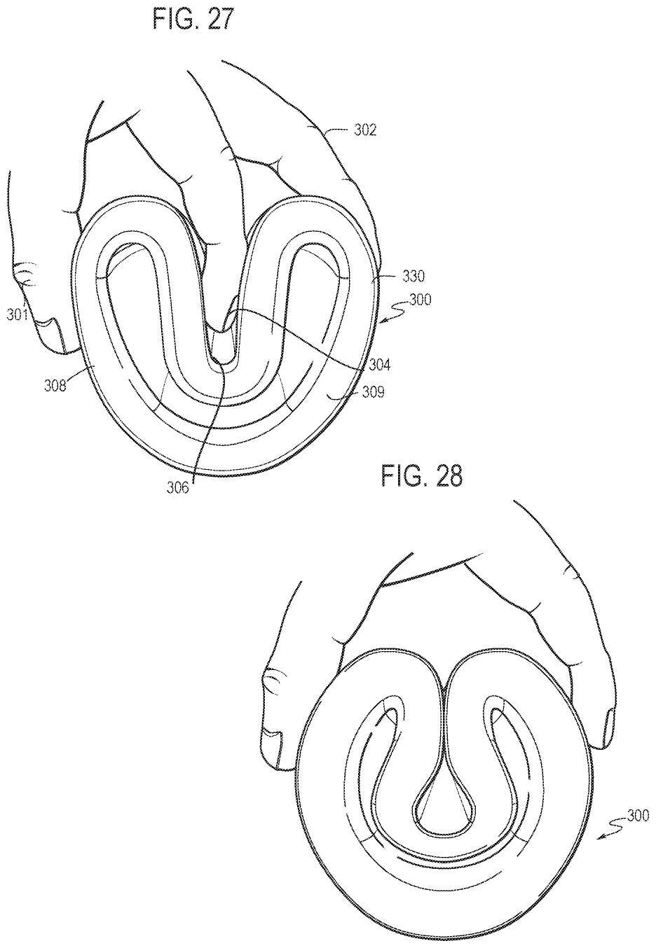

FIGS. 27 and 28 show views of a fluid collection funnel device 300 folded for insertion. As shown, a person grasps the fluid collection funnel between their thumb 301 and their other fingers 302, while pressing their index finger 304 on the pressure point or region on the top/anterior 306 part of the device. As shown, the top or anterior part 306 of the funnel is folded inwardly while the person holds adjacent sides 308, 309 of the device with the thumb 301 and remaining fingers 302. As the index finger 304 is pulled back, the thumb 301 should be brought towards the other fingers 302 in a pinching motion, which forces the device to collapse and be held in a folded position using as few as two fingers, as shown in FIG. 28. The lip 330 is constructed so that a technician can easily fold the funnel device 300 for insertion and also rebound back to an unfolded state after released by the technician.

Once the funnel device 300 is folded, it can be placed within the vagina approximately halfway between the introitus and cervix. The precise location of anchoring may be determined by the individual physician during placement. Once anchoring location is determined, the device is released and allowed to expand to its original shape. The funnel device 300 includes a protruding lip 330 that assists with anchoring the device. Once the funnel device 300 is allowed to unfold within the vaginal canal, the protruding lip 330 engages the vaginal wall.

Immediately after the fluid collection funnel is placed or prior to placement, the collection receptacle is attached to the distal open end of the stem of the funnel via the drainage tubing (if not previously attached). If postpartum hemorrhage is occurring, the blood loss collection device will funnel blood into the tubing and subsequently into the collection receptacle, where total volume of blood loss can be monitored in real time. At 500 ml of blood loss, intervention to begin treatment of a hemorrhage can be considered and 1000 ml of blood loss is considered a critical threshold and requires response. Beyond this threshold of blood loss, the device has performed its function and can be removed for access to the uterus during treatment. The device can be removed from the vagina by inserting two fingers and folding the device before pulling it from the vagina.

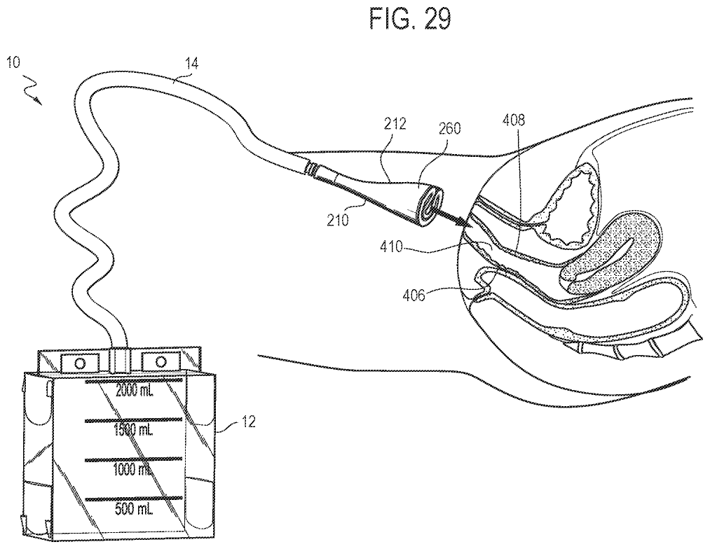

FIG. 29 shows patient anatomy and a system 10 for monitoring post-partum hemorrhaging in the patient prior to insertion of a fluid collection funnel device 260 and FIG. 30 shows patient anatomy and a system 10 for monitoring post-partum hemorrhaging in the patient after insertion of a fluid collection funnel device 260. As shown in FIG. 29, the fluid collection funnel device 260 is connected by drainage tubing 14 to the fluid collection receptacle 12. The funnel 260 device is shown in the compressed configuration in FIG. 29 prior to insertion into the patient anatomy. As shown in FIG. 30, the funnel device 260 is inserted in place into the vaginal canal 410. The contoured anterior surface 212 of the funnel device 260 is disposed against an anterior wall 408 of the vagina 410 and a flattened posterior surface 210 is disposed against a posterior 406 of the vagina 410. When in place, the internal posterior surface 212′ of the funnel device 260 presents an unobstructed flow path.

While various embodiments of the disclosure have been described, the disclosure is not to be restricted except in light of the attached claims and their equivalents. Moreover, the advantages described herein are not necessarily the only advantages of the disclosure and it is not necessarily expected that every embodiment of the disclosure will achieve all of the advantages described. Each of the embodiments described further may include all or only a subset of the features described herein and those of any incorporated material.

What is claimed is:

1. A fluid collection device for a postpartum hemorrhage monitoring system, the device comprising:
a cup comprising an open proximal end, an open distal end, and an interior extending between the open proximal end and the open distal end, the interior configured to receive a bodily fluid, the cup comprising an anterior surface configured for placement adjacent an anterior vaginal wall of a patient and a posterior surface configured for placement adjacent a posterior vaginal wall of a patient;
a ring extending radially exteriorly from the open proximal end of the cup, wherein the ring is incrementally expandable and contractible; and
a stem having an open proximal end, an open distal end, and a lumen extending therebetween, the stem extending between the open distal end of the cup and configured to receive the bodily fluid from the interior of the cup; and
wherein the ring is movable between a collapsed configuration to an expanded configuration upon receiving a fluid within the ring.

2. The device of claim 1, wherein the wherein the anterior surface tapers to the proximal end of the stem and the posterior surface is flush with the stem such that the stem is disposed rearward a geometric center of the cup.

3. The device of claim 1, wherein the cup comprises a pressure point disposed adjacent the ring to facilitate folding of the ring and the cup for placement within a patient.

4. The device of claim 3, wherein the pressure point is an area of the cup that is thinner than a remainder of the cup.

5. The device of claim 1, comprising a pressure point disposed in an area of the cup having a durometer less than a durometer of the remainder of the cup.

6. The device of claim 5, wherein the pressure point is disposed adjacent the ring to facilitate folding of the ring and the cup for placement within a patient.

7. The device of claim 1, comprising an inflation lumen configured to provide inflation fluid to the ring.

8. The device of claim 7, wherein the inflation lumen is integrally formed with the cup.

9. The device of claim 7, wherein the inflation lumen is disposed within a sidewall of the cup, and wherein the cup has a smooth outer surface adjacent the inflation lumen.

10. The device of claim 7, wherein the inflation lumen extends from the ring to one of the open proximal end and the open distal end of the stem.

11. The device of claim 1, wherein the ring comprises a superabsorbent material.

12. The device of claim 11, wherein the ring is configured to expand upon contact with the bodily fluid.

13. The device of claim 1, wherein the ring is an expandable balloon.

14. A postpartum hemorrhage monitoring device comprising:
a cup comprising an open proximal end, an open distal end, and an interior disposed between the open proximal and distal ends, the interior being configured for receiving a bodily fluid, the cup comprising an anterior surface configured for placement adjacent an anterior vaginal wall of a patient and a posterior surface configured for placement adjacent a posterior vaginal wall of a patient;
a ring extending exteriorly from the open proximal end of the cup;
a stem having an open proximal end, an open distal end, and a lumen extending therebetween, the stem extending from the open distal end of the cup and configured to receive the bodily fluid from the interior of the cup;
wherein the ring is incrementally inflatable from a collapsed configuration to a first expanded configuration in response to receiving a fluid within the ring, and is incrementally deflatable from the first expanded configuration to a second configuration upon removal of the fluid from the ring.

15. The device of claim 14, wherein the ring comprises a superabsorbent material.

16. The device of claim 15, wherein the ring is configured to expand upon contact with a bodily fluid.

17. The device of claim 15, wherein the ring is an expandable balloon.

18. The device of claim 15, wherein the ring is asymmetrical.

19. The device of claim 15, wherein the cup comprises a pressure point that is disposed adjacent to the ring to facilitate folding of the ring and the cup for placement within a patient.

20. The device of claim 19, wherein the pressure point comprises an indentation.
